(19) United States
(12) Patent Application Publication
(10) Pub. No.: US 2022/0260736 A1
(43) Pub. Date: Aug. 18, 2022

(54) SILICON PHOTOMULTIPLIERS FOR POSITRON EMISSION TOMOGRAPHY IMAGING SYSTEMS

(71) Applicant: SEMICONDUCTOR COMPONENTS INDUSTRIES, LLC, Phoenix, AZ (US)

(72) Inventor: Nader JEDIDI, Cork (IE)

(73) Assignee: SEMICONDUCTOR COMPONENTS INDUSTRIES, LLC, Phoenix, AZ (US)

(21) Appl. No.: 17/248,985

(22) Filed: Feb. 16, 2021

(51) Int. Cl. G01T 1/29; G01T 1/20; H01L 31/107; H01L 31/0236; H01L 31/0216; A61B 6/00; A61B 6/03

(52) U.S. Cl. CPC G01T 1/2985; G01T 1/2018; H01L 31/107; A61B 6/037; H01L 31/02161; A61B 6/4241; H01L 31/02363

(57) ABSTRACT

A positron emission tomography (PET) imaging system may include a plurality of detector units. Each detector unit may include a scintillator that converts gamma rays to visible light. Each detector unit also includes a sensor with single-photon avalanche diodes (SPADs) such as a silicon photomultiplier. To improve performance of the sensor, a plurality of pyramidal shaped recesses may be formed over each SPAD. The pyramidal shaped recesses may have sidewalls at an angle such that incident light from the scintillator has a higher transmittance to the semiconductor substrate of the sensor.

### BACKGROUND

This relates generally to imaging systems and, more particularly, to positron emission tomography (PET) imaging systems.

Positron emission tomography is a functional imaging technique that uses a radioactive tracer to show how tissues and organs in the human body are functioning. Positron emission tomography has many medical applications. For example, positron emission tomography allows for examination of chemical activity in the body, which may be useful to detect cancer, heart disease, brain disorders, etc.

Positron emission tomography may use an imaging system to sense the position of a radioactive tracer.

It is within this context that the embodiments described herein arise.

### BRIEF DESCRIPTION OF THE DRAWINGS

FIG. 1 is a circuit diagram showing an illustrative single-photon avalanche diode pixel in accordance with an embodiment.

FIG. 2 is a diagram of an illustrative silicon photomultiplier in accordance with an embodiment.

FIG. 3 is a schematic diagram of an illustrative silicon photomultiplier with a fast output terminal in accordance with an embodiment.

FIG. 4 is a diagram of an illustrative silicon photomultiplier comprising an array of microcells.

FIG. 5 is a schematic diagram of an illustrative positron emission tomography (PET) imaging system that includes a SPAD-based semiconductor device in accordance with an embodiment.

FIG. 6 is a cross-sectional side view of an illustrative positron emission tomography (PET) imaging system having a ring of detector blocks in accordance with an embodiment.

FIG. 7 is a cross-sectional side view of an illustrative detector unit for a positron emission tomography (PET) imaging system that includes a scintillator and a SPAD-based semiconductor device in accordance with an embodiment.

FIG. 8 is a graph of frequency versus incidence angle for an illustrative detector unit of the type shown in FIG. 7 in accordance with an embodiment.

FIG. 9 is a graph of transmittance versus incidence angle for illustrative anti-reflective stacks in accordance with an embodiment.

FIG. 10 is a cross-sectional side view of an illustrative detector unit for a positron emission tomography (PET) imaging system that includes a scintillator and a SPAD-based semiconductor device having pyramidal recesses to increase transmittance in accordance with an embodiment.

### DETAILED DESCRIPTION

Embodiments of the present technology relate to imaging systems that include single-photon avalanche diodes (SPADs).

Some imaging systems include image sensors that sense light by converting impinging photons into electrons or holes that are integrated (collected) in pixel photodiodes within the sensor array. After completion of an integration cycle, collected charge is converted into a voltage, which is supplied to the output terminals of the sensor. In complementary metal-oxide semiconductor (CMOS) image sensors, the charge to voltage conversion is accomplished directly in the pixels themselves, and the analog pixel voltage is transferred to the output terminals through various pixel addressing and scanning schemes. The analog pixel voltage can also be later converted on-chip to a digital equivalent and processed in various ways in the digital domain.

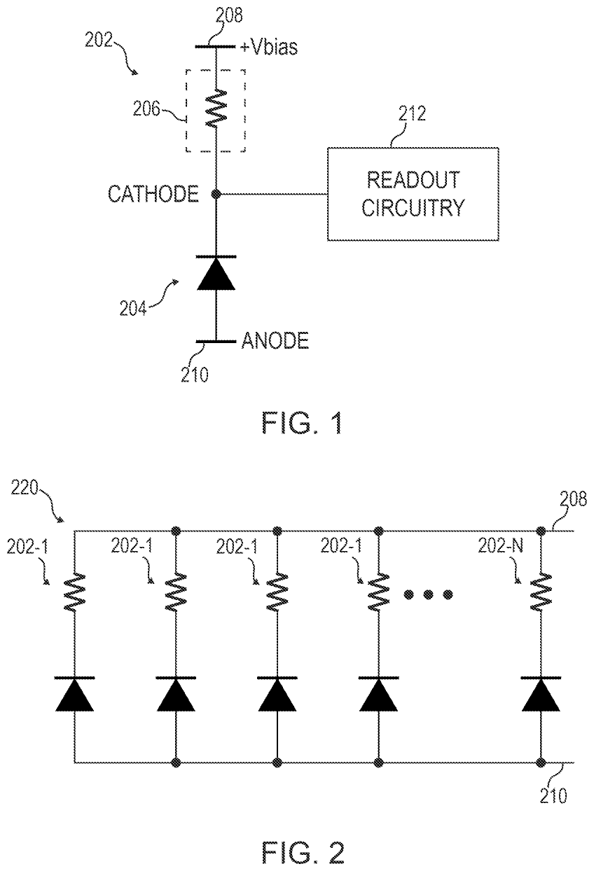

In single-photon avalanche diode (SPAD) devices (such as the ones described in connection with FIGS. 1-4), on the other hand, the photon detection principle is different. The light sensing diode is biased above its breakdown point, and when an incident photon generates an electron or hole, this carrier initiates an avalanche breakdown with additional carriers being generated. The avalanche multiplication may produce a current signal that can be easily detected by readout circuitry associated with the SPAD. The avalanche process can be stopped (or quenched) by lowering the diode bias below its breakdown point. Each SPAD may therefore include a passive and/or active quenching circuit for halting the avalanche.

This concept can be used in two ways. First, the arriving photons may simply be counted (e.g., in low light level applications). Second, the SPAD pixels may be used to measure photon time-of-flight (ToF) from a synchronized light source to a scene object point and back to the sensor, which can be used to obtain a 3-dimensional image of the scene.

FIG. 1 is a circuit diagram of an illustrative SPAD device 202. As shown in FIG. 1, SPAD device 202 includes a SPAD 204 that is coupled in series with quenching circuitry 206 between a first supply voltage terminal 210 (e.g., a ground power supply voltage terminal) and a second supply voltage terminal 208 (e.g., a positive power supply voltage terminal). In particular, SPAD device 202 includes a SPAD 204 having an anode terminal connected to power supply voltage terminal 210 and a cathode terminal connected directly to quenching circuitry 206. SPAD device 202 that includes SPAD 204 connected in series with a quenching resistor 206 is sometimes referred to collectively as a photo-triggered unit or “microcell.” During operation of SPAD device 202, supply voltage terminals 208 and 210 may be used to bias SPAD 204 to a voltage that is higher than the breakdown voltage (e.g., bias voltage Vbias is applied to terminal 208). Breakdown voltage is the largest reverse voltage that can be applied to SPAD 204 without causing an exponential increase in the leakage current in the diode. When SPAD 204 is reverse biased above the breakdown voltage in this manner, absorption of a single-photon can trigger a short-duration but relatively large avalanche current through impact ionization.

Quenching circuitry 206 (sometimes referred to as quenching element 206) may be used to lower the bias voltage of SPAD 204 below the level of the breakdown voltage. Lowering the bias voltage of SPAD 204 below the breakdown voltage stops the avalanche process and corresponding avalanche current. There are numerous ways to form quenching circuitry 206. Quenching circuitry 206 may be passive quenching circuitry or active quenching circuitry. Passive quenching circuitry may, without external control or monitoring, automatically quench the avalanche current once initiated. For example, FIG. 1 shows an example where a resistor component is used to form quenching circuitry 206. his is an example of passive quenching circuitry.

This example of passive quenching circuitry is merely illustrative. Active quenching circuitry may also be used in SPAD device 202. Active quenching circuitry may reduce the time it takes for SPAD device 202 to be reset. This may allow SPAD device 202 to detect incident light at a faster rate than when passive quenching circuitry is used, improving the dynamic range of the SPAD device. Active quenching circuitry may modulate the SPAD quench resistance. For example, before a photon is detected, quench resistance is set high and then once a photon is detected and the avalanche is quenched, quench resistance is minimized to reduce recovery time.

SPAD device 202 may also include readout circuitry 212. There are numerous ways to form readout circuitry 212 to obtain information from SPAD device 202. Readout circuitry 212 may include a pulse counting circuit that counts arriving photons. Alternatively or in addition, readout circuitry 212 may include time-of-flight circuitry that is used to measure photon time-of-flight (ToF). The photon time-of-flight information may be used to perform depth sensing. In one example, photons may be counted by an analog counter to form the light intensity signal as a corresponding pixel voltage. The ToF signal may be obtained by also converting the time of photon flight to a voltage. The example of an analog pulse counting circuit being included in readout circuitry 212 is merely illustrative. If desired, readout circuitry 212 may include digital pulse counting circuits. Readout circuitry 212 may also include amplification circuitry if desired.

The example in FIG. 1 of readout circuitry 212 being coupled to a node between diode 204 and quenching circuitry 206 is merely illustrative. Readout circuitry 212 may be coupled to terminal 208 or any desired portion of the SPAD device. In some cases, quenching circuitry 206 may be considered integral with readout circuitry 212.

Because SPAD devices can detect a single incident photon, the SPAD devices are effective at imaging scenes with low light levels. Each SPAD may detect the number of photons that are received within a given period of time (e.g., using readout circuitry that includes a counting circuit). However, as discussed above, each time a photon is received and an avalanche current initiated, the SPAD device must be quenched and reset before being ready to detect another photon. As incident light levels increase, the reset time becomes limiting to the dynamic range of the SPAD device (e.g., once incident light levels exceed a given level, the SPAD device is triggered immediately upon being reset).

Multiple SPAD devices may be grouped together to help increase dynamic range. FIG. 2 is a circuit diagram of an illustrative group 220 of SPAD devices 202. The group or array of SPAD devices may sometimes be referred to as a silicon photomultiplier (SiPM). As shown in FIG. 2, silicon photomultiplier 220 may include multiple SPAD devices that are coupled in parallel between first supply voltage terminal 208 and second supply voltage terminal 210. FIG. 2 shows N SPAD devices 202 coupled in parallel (e.g., SPAD device 202-1, SPAD device 202-2, SPAD device 202-3, SPAD device 202-4, . . . , SPAD device 202-N). More than two SPAD devices, more than ten SPAD devices, more than one hundred SPAD devices, more than one thousand SPAD devices, etc. may be included in a given silicon photomultiplier 220.

Each SPAD device 202 may sometimes be referred to herein as a SPAD pixel 202. Although not shown explicitly in FIG. 2, readout circuitry for the silicon photomultiplier 220 may measure the combined output current from all of SPAD pixels in the silicon photomultiplier. Configured in this way, the dynamic range of an imaging system including the SPAD pixels may be increased. Each SPAD pixel is not guaranteed to have an avalanche current triggered when an incident photon is received. The SPAD pixels may have an associated probability of an avalanche current being triggered when an incident photon is received. There is a first probability of an electron being created when a photon reaches the diode and then a second probability of the electron triggering an avalanche current. The total probability of a photon triggering an avalanche current may be referred to as the SPAD's photon-detection efficiency (PDE). Grouping multiple SPAD pixels together in the silicon photomultiplier therefore allows for a more accurate measurement of the incoming incident light. For example, if a single SPAD pixel has a PDE of 50% and receives one photon during a time period, there is a 50% chance the photon will not be detected. With the silicon photomultiplier 220 of FIG. 2, chances are that two of the four SPAD pixels will detect the photon, thus improving the provided image data for the time period.

The example of FIG. 2 in which the plurality of SPAD pixels 202 share a common output in silicon photomultiplier 220 is merely illustrative. In the case of an imaging system including a silicon photomultiplier having a common output for all of the SPAD pixels, the imaging system may not have any resolution in imaging a scene (e.g., the silicon photomultiplier can just detect photon flux at a single point). It may be desirable to use SPAD pixels to obtain image data across an array to allow a higher resolution reproduction of the imaged scene. In cases such as these, SPAD pixels in a single imaging system may have per-pixel readout capabilities. Alternatively, an array of silicon photomultipliers (each including more than one SPAD pixel) may be included in the imaging system. The outputs from each pixel or from each silicon photomultiplier may be used to generate image data for an imaged scene. The array may be capable of independent detection (whether using a single SPAD pixel or a plurality of SPAD pixels in a silicon photomultiplier) in a line array (e.g., an array having a single row and multiple columns or a single column and multiple rows) or an array having more than ten, more than one hundred, or more than one thousand rows and/or columns.

While there are a number of possible use cases for SPAD pixels as discussed above, the underlying technology used to detect incident light is the same. All of the aforementioned examples of devices that use SPAD pixels may collectively be referred to as SPAD-based semiconductor devices. A silicon photomultiplier with a plurality of SPAD pixels having a common output may be referred to as a SPAD-based semiconductor device. An array of SPAD pixels with per-pixel readout capabilities may be referred to as a SPAD-based semiconductor device. An array of silicon photomultipliers with per-silicon-photomultiplier readout capabilities may be referred to as a SPAD-based semiconductor device.

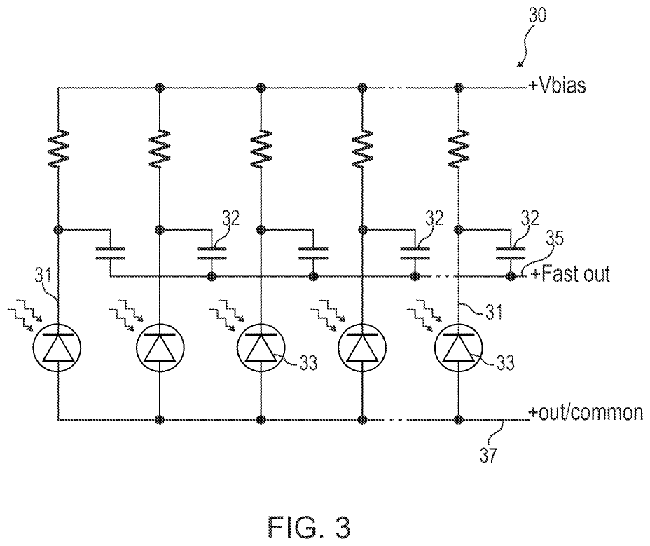

FIG. 3 illustrates a silicon photomultiplier 30. As shown in FIG. 3, SiPM 30 has a third terminal 35 which is capacitively coupled to each cathode terminal 31 in order to provide a fast readout of the avalanche signals from the SPADs 33. When then SPADs 33 emits a current pulse, part of the resulting change in voltage at the cathode 31 will be coupled via the mutual capacitance into the third (“fast”) output terminal 35. Using the third terminal 35 for readout avoids the compromised transient performance resulting from the relatively large RC time constant associated with the biasing circuit that biases the top terminal of the quenching resistor.

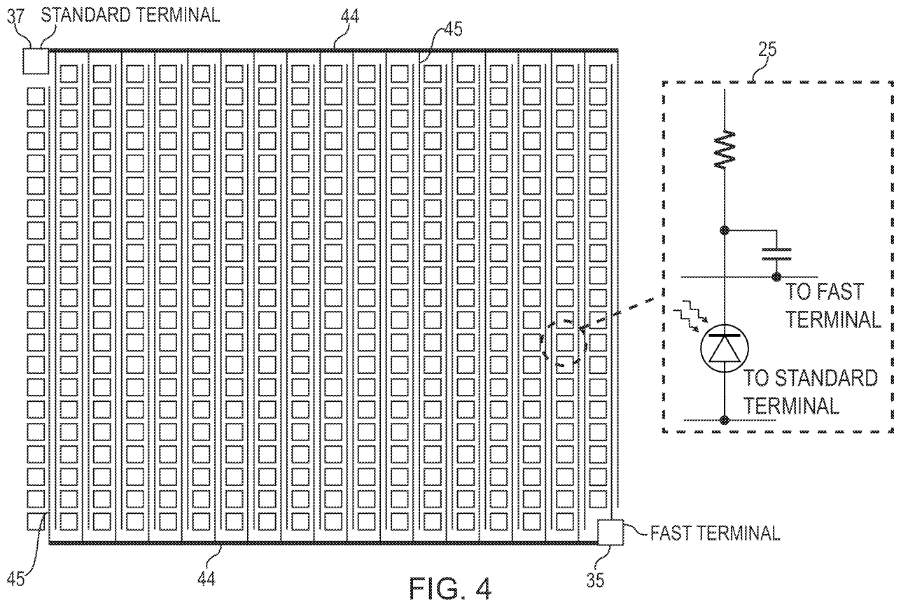

It will be appreciated by those skilled in the art that silicon photomultipliers include major bus lines 44 and minor bus lines 45 as illustrated in FIG. 4. The minor bus lines 45 may connect directly to each individual microcell 25. The minor bus lines 45 are then coupled to the major bus lines 44 which connect to the bond pads associated with terminals 37 and 35. Typically, the minor bus lines 45 extend vertically between the columns of microcells 25, whereas the major bus lines 44 extend horizontally adjacent the outer row of the microcells 25.

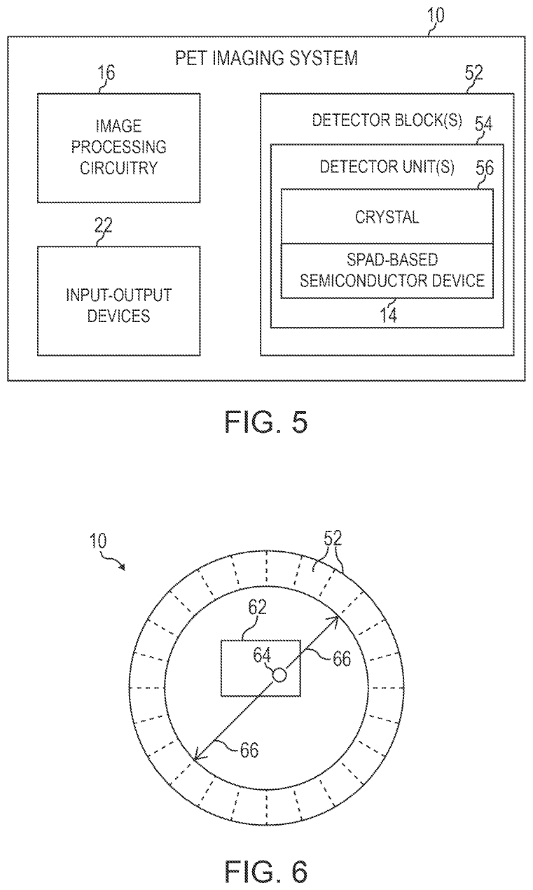

An imaging system 10 with a SPAD-based semiconductor device is shown in FIG. 5. Imaging system 10 may be a medical device such as a positron emission tomography (PET) scanner or other electronic device. Imaging system 10 may sometimes be referred to as a SPAD-based imaging system 10 or a PET imaging system 10.

PET imaging system 10 may include one or more detector blocks 52. Each detector block 52 may include one or more detector units 54. Each detector unit may include a respective SPAD-based semiconductor device 14 (sometimes referred to as semiconductor device 14, device 14, SPAD-based image sensor 14, image sensor 14, silicon multiplier 14, etc.) and crystal 56 (sometimes referred to as scintillator 56). Crystal 56 may absorb ionizing radiation such as gamma rays (e.g., caused by a radioactive tracer used in the PET imaging system) and emit light in the visible spectrum (e.g., blue light).

Crystal 56 may be formed from lutetium-yttrium oxyorthosilicate (LYSO) or any other desired material.

One or more lenses may optionally cover each semiconductor device 14. During operation, lenses (sometimes referred to as optics) may focus light onto scintillator 46 and/or SPAD-based semiconductor device 14. SPAD-based semiconductor device 14 may include SPAD pixels that convert the light into digital data. The SPAD-based semiconductor device may have any number of SPAD pixels (e.g., hundreds, thousands, millions, or more). In some SPAD-based semiconductor devices, each SPAD pixel may be covered by a respective color filter element and/or microlens.

The imaging system may include circuitry such as control circuitry. Each SPAD-based semiconductor device may optionally include respective control circuitry. Alternatively or in addition, each detector block and/or the imaging system may include control circuitry. The control circuitry for each SPAD-based semiconductor device may be formed either on-chip (e.g., on the same semiconductor substrate as the SPAD devices) or off-chip (e.g., on a different semiconductor substrate as the SPAD devices). The control circuitry may control operation of the SPAD-based semiconductor device. For example, the control circuitry may operate active quenching circuitry within the SPAD-based semiconductor device, may control a bias voltage provided to bias voltage supply terminal 208 of each SPAD, may control/monitor the readout circuitry coupled to the SPAD devices, etc.

Each SPAD-based semiconductor device 14 may optionally include additional circuitry such as logic gates, digital counters, time-to-digital converters, bias circuitry (e.g., source follower load circuits), sample and hold circuitry, correlated double sampling (CDS) circuitry, amplifier circuitry, analog-to-digital (ADC) converter circuitry, data output circuitry, memory (e.g., buffer circuitry), address circuitry, etc. Any of the aforementioned circuits may be considered part of the control circuitry.

Image data from SPAD-based semiconductor device 14 may be provided to image processing circuitry 16. Image processing circuitry 16 may be used to perform image processing functions for PET imaging system 10. In some cases, some or all of the control circuitry within PET imaging system 10 may be formed integrally with image processing circuitry 16.

Imaging system 10 may provide a user with numerous high-level functions. To implement these functions, the imaging system may include input-output devices 22 such as keypads, buttons, input-output ports, joysticks, and displays (e.g., touch-sensitive displays). Additional storage and processing circuitry such as volatile and nonvolatile memory (e.g., random-access memory, flash memory, hard drives, solid state drives, etc.), microprocessors, microcontrollers, digital signal processors, application specific integrated circuits, and/or other processing circuits may also be included in the imaging system.

FIG. 6 is a cross-sectional side view of an illustrative PET imaging system 10. As shown, the imaging system may include detector blocks 52 arranged in a ring. The center of the ring is the field-of-view of the imaging system. The subject 62 may be positioned in the center of the ring of detector blocks during operation of imaging system 10.

The subject may be injected with a radiotracer 64. Any desired radiotracer may be used. In general, a radiotracer is formed from a common biological molecule (e.g., glucose, peptides, proteins, etc.) with a radioisotope substituted for one of the components of the molecule. Fluorodeoxyglucose (FDG) is one example of a radiotracer that may be used for PET applications.

After the radiotracer is injected into the subject, the radiotracer arrives at a target organ and participates in the metabolic process of the subject. The radiotracer decays, causing positrons to be generated. The positrons from the radiotracer collide with electrons of neighboring atoms in an annihilation process. The annihilation generates two gamma rays 66.

The scintillators in detector units 54 of detector blocks 52 may absorb the gamma rays 66 and generate light that is then sensed by the SPAD-based semiconductor devices 14. Data from the SPAD-based semiconductor devices may therefore be used to reconstruct a PET image.

The PET imaging system may include any desired number of detector blocks (e.g., 1, more than 1, more than 5, more than 10, more than 20, more than 30, less than 100, less than 50, between 20 and 50, between 25 and 35, etc.). Each detector block may include any desired number of detector units (e.g., 1, more than 1, more than 5, more than 10, more than 30, more than 100, more than 1,000, less than 5,000, less than 1,000, less than 100, less than 30, etc.).

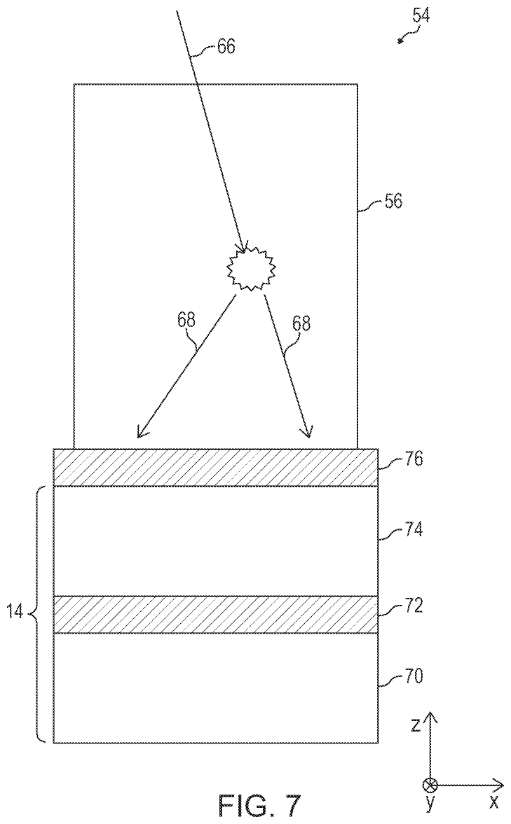

FIG. 7 is a cross-sectional side view of a detector unit 54 that may be included in a PET imaging system. Each detector block 52 in FIG. 6 may include one or more detector units 54 of the type shown in FIG. 7. As shown, the detector unit 54 includes a crystal 56 formed over SPAD-based semiconductor device 14.

An incoming gamma ray 66 (caused by the radiotracer as shown in connection with FIG. 6) may be converted to one or more visible light rays 68 by scintillator 56. The visible light rays 68 are then sensed by SPAD-based semiconductor device 14. SPAD-based semiconductor device 14 includes a SPAD-based sensor 70 that includes one or more single-photon avalanche diodes (SPADs). SPAD -based sensor 70 may be a silicon photomultiplier, as one example. An anti-reflective stack 72 may be formed over silicon photomultiplier 70 (sometimes referred to as sensor 70). The anti-reflective stack may include one or more layers of any desired materials (e.g., silicon nitride, silicon dioxide, etc.). The anti-reflective stack 72 may mitigate reflections of visible light rays to try to maximize the number of generated visible light rays that are detected by SPAD-based sensor 70.

A transparent layer 74 may be formed over the anti-reflective stack. Transparent layer 74 may cover and protect the underlying SPAD-based sensor 70. Transparent layer 74 may sometimes be referred to as cover layer 74 and may be formed from glass, plastic, or any other desired transparent material. An optical grease layer 76 may be interposed between glass layer 74 and scintillator 56. The optical grease may have an index of refraction that is between the indices of refraction of crystal 56 and transparent layer 74. The optical grease layer 76 may ensure that there is no air gap between crystal 56 and transparent layer 74. Optical grease 76 may be formed from any desired material. As one example, optical grease 76 may be formed from an organic material having an index of refraction between 1.4 and 1.6. The index of refraction of optical grease 76 may be greater than the index of refraction of transparent layer 74 (e.g., between 1.3 and 1.5) and less than the index of refraction of crystal 56 (e.g., between 1.7 and 1.9). These examples are merely illustrative. In general, each component may have any desired index of refraction.

The visible light rays incident upon anti-reflective layer 72 tend to have a high angle of incidence. On-axis light may refer to light that travels parallel to the Z-axis in FIG. 7. Light travelling parallel to the Z-axis may be referred to as having an incidence angle of 0 degrees. The higher the angle of the incident light upon anti-reflective layer 72, the more likely the light is to be (undesirably) reflected.

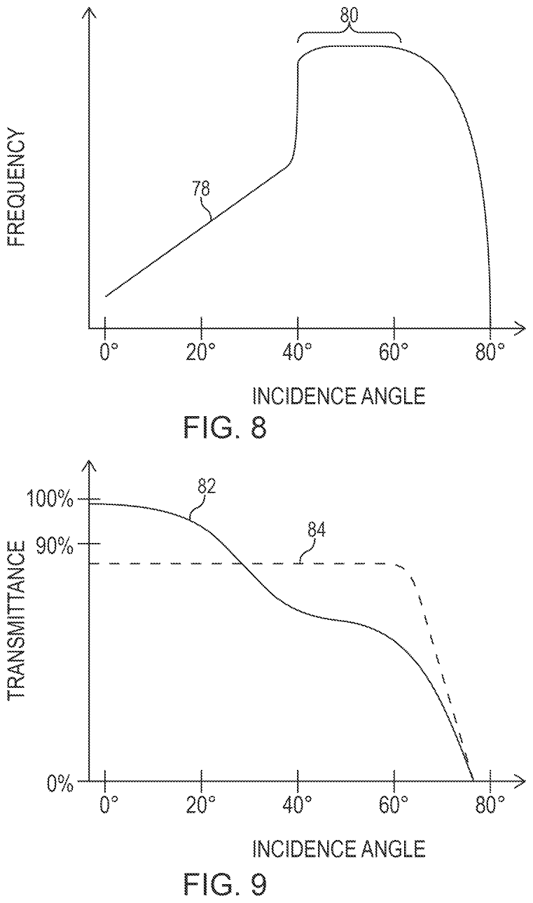

The visible light generated by scintillator 56 may predominantly have a high angle of incidence upon anti-reflective layer 72. FIG. 8 is a graph of frequency versus incidence angle upon anti-reflective stack 72. As shown by frequency profile 78, the frequency of on-axis incident light (e.g., at 0 degrees) is very low. The frequency has a maximum 80 between about 40 degrees and 60 degrees. The frequency may peak at approximately 50 degrees, as one example. The angular distribution of incident light upon anti-reflective stack 72 is therefore centered around 50 degrees (e.g., the average angle of incidence is 50 degrees). More than 70% of photons incident upon anti-reflective stack 72 may have an incidence angle greater than 40 degrees. The average incidence angle may be 50 degrees, between 40 degrees and 60 degrees, between 45 degrees and 55 degrees, between 30 degrees and 70 degrees, etc.

The anti-reflective stack 72 may be optimized based on the type of incident light received. FIG. 9 is a graph of transmittance as a function of incidence angle for different types of anti-reflective stacks. In one possible arrangement, the anti-reflective stack may be optimized for on-axis light (e.g., light that is centered around a 0 degree angle of incidence). This type of anti-reflective stack may have a transmittance profile 82. With this arrangement, the transmittance may be very high (e.g., greater than 90%, greater than 95%, greater than 98%, etc.) at angles between 0 degrees and 20 degrees. However, as the angle of incidence increases the transmittance drops significantly. In PET imaging system 10, where anti-reflective stack 72 receives incident light having a mean angle of incidence of about 50 degrees, the transmittance would be lower than desired.

To improve transmittance in imaging system 10, the anti-reflective stack may be optimized for high angle of incidence light. This type of anti-reflective stack (e.g., a high-angle optimized anti-reflective stack) may have a transmittance profile 84. As shown, the transmittance remains higher across a broader range of angles than in profile 82. However, the maximum transmittance (even at the optimal high angles) is less than 90% with this type of arrangement. This may result in the efficiency of imaging system 10 being lower than desired.

A key metric for PET imaging systems is coincidence resolving time (CRT). Improving the transmittance of light from crystal 56 through anti-reflective stack 72 into SPAD-based sensor 70 improves (reduces) the coincidence resolving time for imaging system 10.

Therefore, to improve performance in imaging system 10, the SPAD-based sensor may include a plurality of pyramids etched into its upper surface. This may ensure that light is incident upon the anti-reflective stack at lower angles, improving transmittance through the anti-reflective stack.

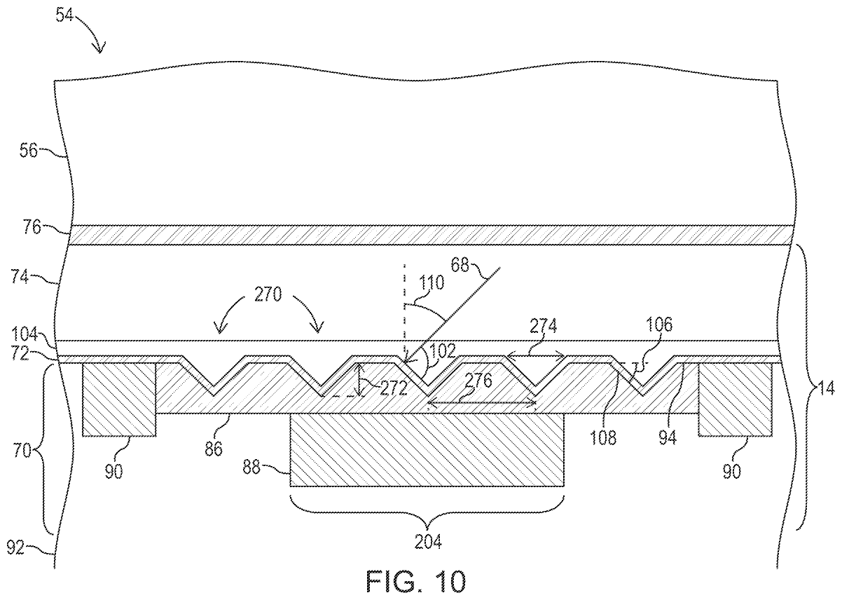

FIG. 10 is a cross-sectional side view of an illustrative detector unit 54 with etched pyramids to increase transmittance. Similar to as shown in FIG. 7, crystal 56 is formed over SPAD-based semiconductor device 14. Optical grease 76 is interposed between transparent layer 74 and crystal 56. The SPAD-based semiconductor device 14 includes a SPAD-based sensor 70 (e.g., a silicon photomultiplier).

As shown in FIG. 10, the silicon photomultiplier 70 may include a single-photon avalanche diode (SPAD) 204 formed in semiconductor substrate 92. There are many ways to form SPAD 204. FIG. 10 shows an example where SPAD 204 includes a doped p+region 86 and an enriched n-type region 88. The SPAD may be surrounded by isolation regions 90. Isolation regions 90 may prevent crosstalk between adjacent SPADs within silicon photomultiplier 70. Isolation regions 90 may be formed from local oxidation of silicon (LOCOS) regions. This example is merely illustrative. In general, isolation regions 90 may be formed from any desired material(s).

Semiconductor layer 92 (used to form sensor 70) has an upper surface 94. As shown in FIG. 10, recesses 270 are formed in the upper surface 94 of semiconductor layer 92. Recesses 270 (sometimes referred to as pyramid-shaped structures 270, transmittance-increasing recesses 270, etc.) may have sidewalls 108 that are angled relative to planar upper surface 94 of semiconductor substrate 92. Anti-reflective stack 72 conforms to the shape of recesses 270. Anti-reflective stack 72 may have a uniform thickness across the upper surface of semiconductor substrate 92 (e.g., the thickness inside and outside of the recesses is the same).

Because of the presence of recesses 270 (which include angled sidewalls), incident light 68 (which approaches upper surface 94 at high angles) is incident upon anti-reflective stack 72 at an angle 102 that is close to 90 degrees (e.g., close to on-axis).

A planarization layer 104 may also be formed in recesses 270. Planarization layer 104 may be formed from silicon dioxide or any other desired material. Planarization layer 104 is interposed between anti-reflective stack 72 and transparent layer 74.

Recesses 270 may have any desired size and shape. Recesses 270 may be pyramid shaped structures (e.g., rectangular-based pyramids such as square-based pyramids, diamond-based pyramids, triangular-based pyramids, etc.). The recesses may be defined by sidewalls 108 that are etched into semiconductor substrate 92. Sidewalls 108 may be at an angle 106 relative to the planar upper surface 94 of semiconductor substrate 92. Angle 106 may be between 30 degrees and 70 degrees, between 40 degrees and 60 degrees, between 45 degrees and 55 degrees, between 50 degrees and 55 degrees, greater than 30 degrees, less than 70 degrees, etc.

As discussed above, the average angle 110 of incoming light 68 relative to on-axis light (e.g., 0 degree light) may be 50 degrees, between 40 degrees and 60 degrees, between 45 degrees and 55 degrees, between 30 degrees and 70 degrees, etc.

The difference between angle 106 of the recess-defining sidewall and the average angle 110 of incident light 68 may be less than 20 degrees, less than 10 degrees, less than 5 degrees, less than 3 degrees, less than 1 degree, etc. Minimizing this difference results in incident light 68 reaching the sidewalls 108 at angle 102 that may be between 80 degrees and 100 degrees, between 70 degrees and 110 degrees, etc. In other words, the average angle of incidence for light 68 upon sidewalls 108 is within 20 degrees, within 10 degrees, within 5 degrees, within 3 degrees, within 1 degree, etc. of the surface normal for sidewalls 108 (e.g., on-axis for sidewalls 108).

Because pyramidal recesses 270 cause incident light to be incident at on-axis angles relative to the anti-reflective stack (instead of off-axis angles), the on-axis optimized anti-reflective stack 72 (e.g., having transmittance profile 82 in FIG. 9) may be used. Accordingly, the transmittance through the anti-reflective stack for the average incidence angle may be high (e.g., greater than 90%, greater than 95%, greater than 98%, etc.).

Recesses 270 may be formed using trenches (e.g., etched trenches) or using any other desired structures/techniques. The trenches may extend from surface 94 (e.g., an upper surface) towards the opposing surface (e.g., a lower surface) of semiconductor layer 92. The recesses each have a height 272 (sometimes referred to as depth) and a width 274. The recesses also have a pitch 276 (e.g., the center-to-center separation between each recess). In general, each recess may have a height 272 of less than 5 micron, less than 3 micron, less than 2 micron, less than 1 micron, less than 0.5 micron, less than 0.2 micron, less than 0.1 micron, greater than 0.01 micron, greater than 0.5 micron, greater than 1 micron, between 1 and 2 micron, between 0.5 and 3 micron, between 0.3 micron and 10 micron, between 0.01 micron and 0.2 micron, etc. Each recess may have a width 274 of less than 5 micron, less than 3 micron, less than 2 micron, less than 1 micron, less than 0.5 micron, less than 0.2 micron, less than 0.1 micron, greater than 0.01 micron, greater than 0.5 micron, greater than 1 micron, between 1 and 2 micron, between 0.5 and 3 micron, between 0.3 micron and 10 micron, between 0.01 micron and 0.2 micron, etc. The pitch 276 may be less than 5 micron, less than 3 micron, less than 2 micron, less than 1 micron, less than 0.5 micron, less than 0.3 micron, less than 0.1 micron, greater than 0.01 micron, great than 0.1 micron, greater than 0.5 micron, greater than 1 micron, between 1 and 2 micron, between 0.5 and 3 micron, between 0.3 micron and 10 micron, between 0.01 and 0.3 micron, between 0.01 micron and 1 micron, etc.

The ratio of the width 274 to the pitch 276 may be referred to as the duty cycle or the etch percentage for the substrate. The duty cycle (etch percentage) indicates how much unetched substrate is present between each pair of recesses and how much of the upper surface of the substrate is etched to form the recesses. The ratio may be 100% (e.g., each recess is immediately adjacent to surrounding recesses), lower than 100%, lower than 90%, lower than 70%, lower than 60%, greater than 50%, greater than 70%, between (and including) 50% and 100%, etc. The semiconductor substrate 92 may have a thickness of greater than 4 micron, greater than 6 micron, greater than 8 micron, greater than 10 micron, greater than 12 micron, less than 12 micron, between 4 and 10 micron, between 5 and 20 micron, less than 10 micron, less than 6 micron, less than 4 micron, less than 2 micron, greater than 1 micron, etc.

Each SPAD may be covered by any desired number of recesses 270 (e.g., 1 recess, more than 1 recess, more than 4 recesses, more than 9 recesses, more than 25 recesses, more than 50 recesses, more than 100 recesses, more than 300 recesses, more than 1,000 recesses, less than 1,000 recesses, less than 300 recesses, less than 100 recesses, less than 50 recesses, less than 25 recesses, etc.

The foregoing is merely illustrative of the principles of this invention and various modifications can be made by those skilled in the art. The foregoing embodiments may be implemented individually or in any combination.

1. A semiconductor device configured to operate in a positron emission tomography imaging system that includes a scintillator, the semiconductor device comprising:
a semiconductor substrate having an upper surface that is configured to receive incident light from the scintillator of the positron emission tomography imaging system;
a single-photon avalanche diode in the semiconductor substrate; and
a plurality of pyramidal recesses in the upper surface of the semiconductor substrate, wherein the single-photon avalanche diode is overlapped by the plurality of pyramidal recesses.

2. The semiconductor device defined in claim 1, further comprising:
an anti-reflective layer that is formed over the upper surface of the semiconductor substrate.

3. The semiconductor device defined in claim 2, wherein the anti-reflective layer conforms to the plurality of pyramidal recesses.

4. The semiconductor device defined in claim 2, further comprising:
a transparent cover layer, wherein the anti-reflective layer is interposed between the transparent cover layer and the semiconductor substrate.

5. The semiconductor device defined in claim 4, wherein the transparent cover layer is interposed between the anti-reflective layer and an optical grease layer.

6. The semiconductor device defined in claim 4, further comprising:
a planarization layer, wherein the planarization layer is interposed between the transparent cover layer and the anti-reflective layer.

7. The semiconductor device defined in claim 1, wherein the incident light from the scintillator has an average angle of incidence between 30 degrees and 70 degrees relative to a surface normal of a planar portion of the upper surface of the semiconductor substrate.

8. The semiconductor device defined in claim 7, wherein each one of the plurality of pyramidal recesses is defined by a sidewall that is at an angle relative to the planar portion of the upper surface of the semiconductor substrate and wherein the angle is between 30 degrees and 70 degrees.

9. The semiconductor device defined in claim 8, wherein a difference between the average angle of incidence of the incident light from the scintillator and the angle of the sidewall is less than 20 degrees.

10. The semiconductor device defined in claim 8, wherein a difference between the average angle of incidence of the incident light from the scintillator and the angle of the sidewall is less than 10 degrees.

11. The semiconductor device defined in claim 8, wherein the incident light from the scintillator has an average angle of incidence on the sidewalls of the pyramidal recesses that is within 10 degrees of a surface normal for the sidewalls.

12. A semiconductor device configured to operate in a positron emission tomography imaging system that includes a scintillator, comprising:
a semiconductor substrate having an upper surface;
a single-photon avalanche diode in the semiconductor substrate; and
recesses in the upper surface of the semiconductor substrate, wherein the recesses overlap the single-photon avalanche diode; and
an anti-reflective layer that is formed over the upper surface of the semiconductor substrate and conforms to the recesses, wherein the recesses are defined by sidewalls at non-zero angles relative to the upper surface of the semiconductor substrate and wherein incident light from the scintillator has an average angle of incidence on the sidewalls of the recesses that is within 10 degrees of a surface normal for the sidewalls.

13. The semiconductor device defined in claim 12, further comprising:
a transparent cover layer, wherein the anti-reflective layer is interposed between the transparent cover layer and the semiconductor substrate.

14. The semiconductor device defined in claim 13, wherein the transparent cover layer is configured to be interposed between an optical grease layer and the anti-reflective layer.

15. The semiconductor device defined in claim 14, further comprising:
a planarization layer, wherein the planarization layer is interposed between the transparent cover layer and the anti-reflective layer.

16. The semiconductor device defined in claim 12, wherein the upper surface has a planar portion and wherein the average angle of incidence relative to a surface normal of the planar portion is between 30 degrees and 70 degrees.

17. (canceled)

18. A positron emission tomography imaging system that includes a detector unit, wherein the detector unit comprises:
a scintillator;
a sensor that is adjacent to the scintillator,
wherein the sensor comprises:
a semiconductor substrate that is configured to receive incident light from the scintillator, wherein the semiconductor substrate is formed from a semiconductor material with an upper surface;
a single-photon avalanche diode in the semiconductor substrate; and
a plurality of pyramidal recesses that extend into the upper surface of the semiconductor material and that overlap the single-photon avalanche diode.

19. The positron emission tomography imaging system defined in claim 18, wherein the incident light from the scintillator has an average angle of incidence between 30 degrees and 70 degrees relative to a surface normal of a planar portion of the upper surface of the semiconductor substrate, wherein each one of the plurality of pyramidal recesses is defined by a sidewall that is at an angle relative to the planar portion of the upper surface of the semiconductor substrate, and wherein the angle is between 30 degrees and 70 degrees.

20. The positron emission tomography imaging system defined in claim 18, wherein the scintillator is configured to convert gamma rays into visible light.
